(19) United States
(12) Patent Application Publication
(10) Pub. No.: US 2009/0187163 A1
(43) Pub. Date: Jul. 23, 2009

(54) Controlled Stiffness Guidewire Unit

(76) Inventor: Bernhard Uihlein, Dettingen (DE)

Correspondence Address: CROWELL & MORING LLP;INTELLECTUAL PROPERTY GROUP, P.O. BOX 14300, WASHINGTON, DC, 20044-4300, US

(21) Appl. No.: 12/300,615

(22) PCT Filed: May 15, 2007
(86) PCT No.: PCT/EP2007/004301

§ 371 Date: Nov. 12, 2008

(51) Int. Cl. A61M 25/09

(52) U.S. Cl. 604/528

(57) ABSTRACT

A guide wire unit includes a section which can be stiffened in a manner controlled by a pressure medium. The section has a pressure channel for the controlled production of a stiffening pressure by introducing a pressure medium, and flexible stiffening elements that are forced one against the other in a manner increasing the stiffening effect when subjected to a pressure load by the effect of the stiffening pressure. The flexible stiffening elements have an inner stiffening tube part and a radially less flexible outer stiffening tube part surrounding the same. Under the effect of the stiffening pressure, the inner stiffening tube part is forced against the interior of the outer stiffening tube part, thereby increasing the stiffening effect. A distal end section can be mounted distally and/or a stiff section can be mounted proximally in front of the controlled stiffness section. A center guide wire core may extend in the controlled stiffness section. The guide wires are usable as guide wires for positioning medical catheters.

### CROSS-REFERENCE TO RELATED APPLICATIONS

This application contains related subject matter to co-pending application Ser. No. ______, which is a national stage of PCT International Application No. PCT/EP2007/004410, filed May 16, 2007 and designating the United States.

### BACKGROUND AND SUMMARY OF THE INVENTION

The invention relates to a guide wire unit and, in particular, a guide wire unit for medical instruments. Such a guide wire unit has a section which can be stiffened in a manner controlled by a pressure medium. This means that, in a manner controlled by the pressure medium, this section can optionally be changed into one of several different stiffness conditions respectively, in which case the section may extend over a smaller or greater part of the entire length of the guide wire unit until it reaches the extreme case in which it makes up the entire length of the guide wire unit. Guide wire units are used, for example, for medical instruments, such as the positioning of catheters in a patient's body passages and tissue ducts.

Specifically also for medical applications of this type, it is known to design various axial sections of a guide wire unit with predefined different stiffnesses, particularly to design a distal end section to be more flexible than a proximally adjoining shaft section leading to a rear operating/grip area. This is implemented, for example, in that a guide wire core is tapered in the distal, i.e., application-side forward end section and/or is surrounded with a softer covering than in the shaft section. Several guide wires of this type are described, for example, in German patent document DE 101 38 953 B4.

In addition to guide wire units which can be stiffened in a magnetically controlled manner, International Published Patent Application WO 2004/035124 A1 also discloses a guide wire unit having three wire strands of an essentially identical, partially cylindrical cross-section, which can be mutually engaged in a stiffness-increasing manner on surface areas pointing to one another, for the purpose of which they are provided there with a toothing, for example, in the form of fine hairs. For separating the surface areas from one another, a pressure medium is introduced into gaps between these surface areas, so that the guide wire unit reaches a condition of low stiffness. In order to return it to a condition of higher stiffness, the pressure medium is withdrawn, preferably while additionally generating a vacuum. Furthermore, a guide wire unit of the above-mentioned type is described there, which is constructed of a central flexible tube, of an inflexible or barely flexible outer cover coaxially surrounding the flexible tube while forming an annular gap, and of several axially extending wire strands arranged in the annular gap in a manner distributed in the circumferential direction. By introducing a pressure medium into the interior of the central tube, the latter presses the wire strands radially against the outer cover, whereby the guide wire unit assumes a condition of a higher bending resistance.

The invention is based on the technical problem of providing a guide wire unit of the above-mentioned type, which is further improved with respect to the above-explained state of the art, and particularly offers functional and/or manufacturing-related advantages with respect to the latter.

The invention solves this problem by providing a guide wire unit particularly for medical instruments, having a section which can be stiffened in a pressure-medium-controlled manner and has a pressure channel for the controllable generating of a stiffening pressure by introducing a pressure medium, and flexible stiffening elements which, under a pressure load, when subjected to the stiffening pressure, are pressed against one another increasing the stiffening effect. The flexible stiffening elements comprise an inner stiffening tube part and an outer stiffening tube part, which surrounds the inner stiffening tube part and is radially less flexible with respect to the latter, under the effect of the stiffening pressure. The inner stiffening tube part is pressed against the interior side of the outer stiffening tube part thereby increasing the stiffening effect.

In the case of the guide wire unit according to the invention, the outer stiffening tube part and inner stiffening tube part form the flexible stiffening elements which, when subjected to a pressure load, are pressed against one another in a manner increasing the stiffening in order to change the corresponding section, which can be stiffened in a manner controlled by a pressure medium, into a condition of higher stiffness. It is demonstrated that this implementation offers high functional and manufacturing-related advantages. As a result of the principle, a large-surface pressure contact of the inner stiffening tube part with its exterior side against the interior side of the outer stiffening tube part in the pressure-loaded condition is obtained, so that a higher bending resistance is provided correspondingly uniformly over the entire section that can be stiffened in a controlled manner and for all bending directions. In addition, suitable tube parts can be produced in a relatively easily. Thus, as a further development of the invention, the outer stiffening tube part and/or the inner stiffening tube part consists of a tube braiding which, in particular, may be a wire braiding which is conventional per se.

In addition to the section that can be stiffened in a pressure-medium-controlled manner, the guide wire unit may contain one or more additional sections which are fixedly defined with respect to their stiffness behavior. On the one hand, a distal flexible end section may be disposed distally directly, or with the insertion of one or more other sections, in front of the section which can be stiffened in a manner controlled by a pressure medium. In addition or as an alternative, a stiff section may be disposed proximally directly or with the insertion of one or more other sections in front of the section which can be stiffened in a manner controlled by a pressure medium, which stiff section contains a pressure-resistant tube for guiding the pressure medium through the section which can be stiffened in a manner controlled by a pressure medium. Therefore, in the case of this implementation, the variable pressure-medium-controlled stiffness adjustment is limited to the corresponding part of the entire length of the guide wire unit, while the guide wire unit in its distal end region and/or in a shaft area proximally disposed in front, can specifically be designed for other requirements, as they are customary for guide wires; for example, for specific designs of the distal end area as a work/work piece area.

In the case of a further developed guide wire unit, a central guide wire core extends in the section that can be stiffened in a manner controlled by a pressure medium, and the pressure channel fitting the latter is constructed as an annular channel, which radially on the outside adjoins the central guide wire core. This implementation has the advantage that, as a result of the central guide wire core, a corresponding basic stiffness can be adjusted for the section that can be stiffened in a manner controlled by a pressure medium, and, as required, a one-piece continuous central guide wire core can be used for the guide wire unit in cases in which the guide wire unit has one or more additional sections in addition to the section that can be stiffened in a manner controlled by a pressure medium and a central guide wire core is desirable also at least in one of these other sections for functional or other reasons.

As a further development of the invention, the flexible stiffening elements are designed for the frictionally engaged and/or form-fitting stiffening interaction, for which functionally reliable solutions that are simple with respect to manufacturing are available.

In an advantageous further development of the invention, the pressure channel is radially bounded by a surrounding, radially flexible tube membrane which, radially on the outside, is adjoined by flexible stiffening elements. The tube membrane causes a fluid-tight action upon the flexible stiffening elements by way of a radially outwardly acting pressure force under the effect of the stiffening pressure, so that, as desired, the flexible stiffening elements are pressed against one another while increasing the stiffening.

In another embodiment of the invention, the flexible stiffening elements have an outer sleeve and at least one inner member arranged between the tube membrane and the outer sleeve, which inner member, under the effect of the radially outward-pointing pressure force component, is pressed against the outer sleeve in order to thereby cause the stiffness-increasing condition, for example, by frictional engagement and/or form fitting with the outer sleeve.

In a further embodiment, several inner members are provided which, extending with an axial direction component in the circumferential direction, are arranged in a distributed manner between the tube membrane and the outer sleeve. Here, in a further development, the inner members may be made, for example, of a flat-wire or round-wire material or of a strand material or rope material, or the at least one inner member is implemented as a tube braiding which can be radially stretched as required. Corresponding flat or round wires, strands, ropes or tube braidings, which act as flexible inner members, may, in particular, extend along the entire or at least the predominant length of the section, which can be stiffened in a manner controlled by a pressure medium, in the axial direction and thereby absorb tensile forces and, in this manner, stabilize the longitudinal course of the outer sleeve and thereby of the section, which can be stiffened in a manner controlled by the pressure medium, on the whole, also in curved areas in a stiffening manner. In one embodiment of the invention, the outer sleeve is formed by an outer stiffening tube part.

In a further development of the invention, the end section disposed distally in front of the section which can be stiffened in a controlled manner has a central distal guide wire core and a covering surrounding the latter. In this manner, the distal end section can be conventionally designed for the demands made on it. In a further development, the covering of the distal end section contains a plastic covering, a coil spring, and/or a tube braiding. In corresponding embodiments, the distal guide wire core together with the central guide wire core of the section which can be stiffened in a controlled manner is formed in one piece of a single guide wire core which extends at least in these two sections of the guide wire unit.

In a further development of the invention, the pressure-resistant tube of the stiff section proximally disposed in front of the section that can be stiffened in a controlled manner is surrounded by a covering containing a coil spring and/or a tube braiding. As a result, the guide wire unit can also be optimized in its design in this area.

In a further development of the invention, the tube braiding of the covering in the distal end section and/or the tube braiding of the covering in the stiff section proximally disposed in front is formed in one piece with the tube braiding of the outer stiffening tube part or the outer sleeve and/or with the tube braiding of the inner stiffening part of the section that can be stiffened in a controlled manner. This measure has advantages related to manufacturing and, in addition, permits a uniform surface design of the guide wire unit in the corresponding sections.

In an advantageous further development of the invention, a proximal pressure medium connection device with a pressure medium connection tube is provided into which a check valve is inserted and/or which is releasably in a fluid-tight manner held by a tube holding device, which can be activated by the admission of pressure, in a connection body. As a result, the guide wire unit with the section that can be stiffened in a manner controlled by a pressure medium can be releasably held, for example, by use of a connection body of an otherwise conventional construction, for example, a connection body of the Luer type which is customary in medical applications.

In an advantageous embodiment, the tube holding device contains a sealing membrane radially surrounding the pressure medium connection tube, which sealing membrane is adjoined radially on the outside in the connection body by an annular pressure channel. As a result of the action of the pressure on this annular pressure channel, the membrane can firmly place itself against the pressure medium connection tube in order to hold the latter in a secure and fluid-tight manner. In a further embodiment of the invention, it is advantageously provided that the annular pressure channel can be acted upon by the same pressure medium as the section that can be stiffened in a controlled manner, so that only a single pressure medium and a single pressure source respectively are required.

Advantageous embodiments of the invention are illustrated in the drawings and will be described in the following.

### BRIEF DESCRIPTION OF THE DRAWINGS

FIG. 1 is a longitudinal sectional view of a first guide wire unit with a proximal connection area, a proximal stiff section, a section that can be stiffened in a manner controlled by a pressure medium, and a distal end section;

FIG. 2 is a cross-sectional view along Line II-II of FIG. 1;

FIG. 3 is a cross-sectional view along Line III-III of FIG. 1;

FIG. 4 is a cross-sectional view along Line IV-IV of FIG. 1;

FIG. 5 is a cutout-type half-section longitudinal view of the guide wire unit of FIG. 1 in the section that can be stiffened in a manner controlled by pressure;

FIG. 6 is a longitudinal sectional view of a second guide wire unit with a proximal stiff section and a distal end section both modified with respect to FIG. 1;

FIG. 7 is a cross-sectional view along Line VII-VII of FIG. 6;

FIG. 8 is a cross-sectional view along Line VIII-VIII of FIG. 6;

FIG. 9 is a longitudinal sectional view of a third guide wire unit with a distal end section modified with respect to FIG. 6;

FIG. 10 is a cross-sectional view along a line X-X of FIG. 9;

FIG. 11 is a longitudinal sectional view of a fourth guide wire unit having a distal end region which is modified with respect to FIG. 9;

FIG. 12 is a cross-sectional view along a Line XII-XII of FIG. 11; and

FIG. 13 is a longitudinal sectional view of the guide wire unit of FIG. 6 having a coupled connection body.

### DETAILED DESCRIPTION OF THE DRAWINGS

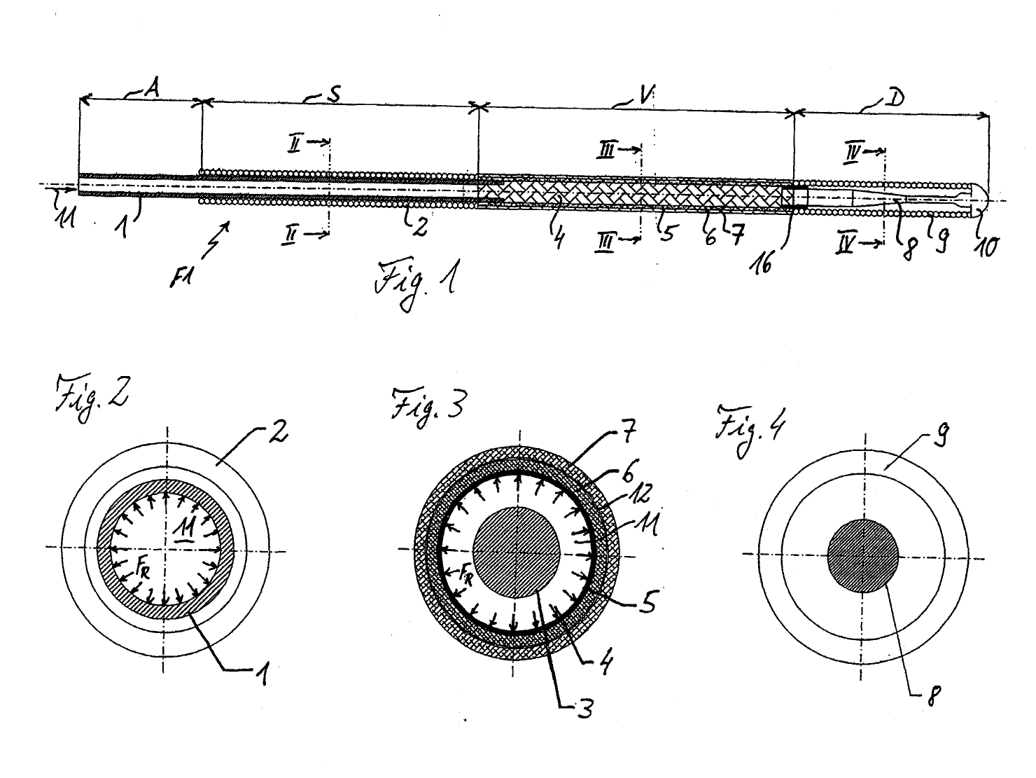

In the following, several selected embodiments of guide wire units according to the invention will be explained in detail with reference to the drawings. For an easier understanding, the same or similar reference symbols are used for elements which are identical or functionally equivalent in the different examples. From a proximal rearward end to a distal forward end, a first guide wire unit F1 illustrated in FIG. 1 successively has a connection section A, a stiff section S, a section V which can be variably stiffened in a controlled manner, and a distal end section D. For the purpose of improved clarity, the different section A, S, V, D in FIG. 1 have similar lengths which in reality may, however, clearly differ from one another, depending on the demand and usage case. Thus, particularly section V, which can be variably stiffened in a controlled manner, in corresponding embodiments, may have a clearly greater length than the other sections A, S, D and may make up, for example, much more than half of the entire length of the guide wire unit F1.

A stiff pressure tube 1 extends in the connection section A and in the stiff section S, the stiff section S differing from the connection section A in that there the stiff pressure tube 1 is surrounded by a coil spring 2, as also illustrated in the cross-sectional view of FIG. 2.

In section V, which can be stiffened in a controlled manner, the guide wire unit F1, as illustrated particularly in the cross-sectional view of FIG. 3, has an optional guide wire core or stiffening core 3, which is radially surrounded by an annular channel 4 acting as a pressure channel which, in turn, is bounded radially to the outside in a fluid-tight manner by a flexible, stretchable tube membrane 5. Flexible stiffening elements adjoin the tube membrane 5 radially on the outside, which stiffening elements in this example are formed by an inner stiffening tube part 6 and an outer stiffening tube part 7 surrounding the latter.

In this example, the inner stiffening tube part 6 and the outer stiffening tube part 7 are each formed by a braided tube, especially by a braided wire tube, of a type customary per se, as shown more clearly in the cutout-type semi-sectional view of FIG. 5. Especially FIG. 5 also demonstrates that, in this embodiment, the inner stiffening part 6 has an axial braiding component which is more pronounced in comparison with the outer stiffening tube part 7 and a smaller braiding component in the circumferential direction, so that it can absorb occurring axial tensile stress and is more flexible with respect to radial compressive stress than the outer stiffening tube part 7.

In a manner that is conventional per se, the distal end section D is implemented by a distal guide wire core 8, which tapers in the distal direction and widens in the manner of a blade at the distal end, and by a coil spring 9 enveloping the guide wire core 8. At the distal end, the distal end section D is closed off with an atraumatic hemispherical end body 10, which is adjoined flush on the outside by the coil spring 9.

Depending on the use, the distal guide wire core 8 and the guide wire core 3 in section V, which can be stiffened in a controlled manner, may be two separate parts or may be formed by a guide wire core which continues in one piece. In other embodiments, the optional guide wire core 3 is not used in section V which can be stiffened in a controlled manner. The pressure channel 4 will then take up the entire interior of the tube membrane 5.

Section V, which can be variably stiffened in a controlled manner, is especially a section of the guide wire unit F which, while being controlled by use of a pressure medium 11, can be changed into conditions of different stiffness. For this purpose, the pressure medium 11 is fed from the proximal end into the stiff pressure tube 1 and, in it, is conducted to section V, which can be stiffened in a controlled manner, where it reaches the pressure channel 4 bounded by the radially flexible tube membrane 5. The stiffening pressure, especially an excess pressure, generated by the introduced pressure medium 11, results in radially outwardly acting pressure force FR, which has no effect in the pressure tube 1 because of its high stiffness but, in the section V which can be stiffened in a controlled manner, elastically expands the tube membrane 5 radially toward the outside. The latter has the result that the radially slightly flexible inner stiffening tube part 6 is pressed with its outer side firmly against the inner side of the less flexible outer stiffening tube part 7. A quasi-flat frictional engagement and/or form-fitting is thus caused between the two tube wire braidings 6, 7 in a pertaining transition/boundary surface region 12. The quasi-flat frictional engagement/form-fitting between the inner and the outer stiffening tube part 6, 7, in contrast to the pressure-relieved condition in which the inner stiffening tube part 6 is surrounded by the outer stiffening tube part with a loose or low contact, causes a clear stiffness increase for section V, which can be stiffened in a controlled manner.

Depending on the system design, it may be provided that section V, which can be stiffened in manner controlled by the pressure medium in this fashion, can be changed between a single pressure-relieved condition of low stiffness and a single pressure-loaded condition of high stiffness, or several pressure-loaded conditions with different stiffening pressures of the pressure medium 11 and correspondingly higher stiffnesses can be implemented, as a result of the principle, the stiffness of section V, which can be stiffened in a controlled manner, also increasing with a higher stiffening pressure.

In contrast to section V, which can be stiffened in a controlled manner, the distal end section D is closed off in a fluid-tight manner. For this purpose, a cylindrical connection piece 16 is provided at the transition between section V, which can be stiffened in a controlled manner, and the distal end section D. On the distal side of this connection piece 16, the distal guide wire core 8 is held and/or fitted through, while the tube membrane is placed in a fluid-tight manner on the proximal side of the connection piece 16, for example, by shrink fitting. As required, the fluid tightness of the connection piece 16 can be ensured by the inserted distal guide wire core 8 and/or in that a solid cylinder body is selected for the connection piece 16.

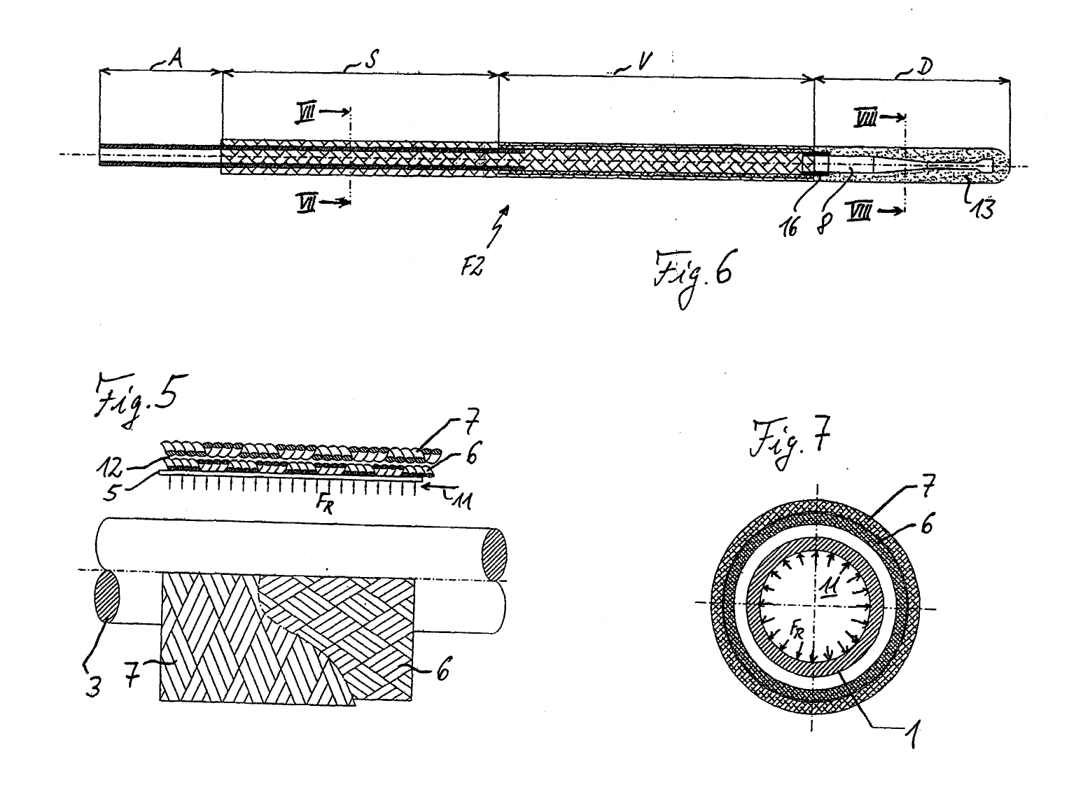

Instead of the inner stiffening tube part and the outer stiffening tube part, other implementations of flexible stiffening elements are also contemplated which, in the case of a pressure load under the effect of the stiffening pressure, for example, by the radial expansion of the tube membrane 5, are pressed against one another in a stiffness-increasing manner. Thus, the flexible stiffening elements may contain, for example, an outer sleeve and at least one inner member arranged between the tube membrane and the outer sleeve, which inner member, in the case of a pressure load, is pressed by the tube membrane against the flexible outer sleeve. In this case, several inner members extending with an axial direction component may be arranged distributed in the circumferential direction between the tube membrane and the outer sleeve, in which case, the at least one inner member can be formed, for example, of a flat or round material or strand material or rope material. The at least one inner member may be prefabricated together with the tube membrane on its outer side or together with the outer sleeve on its inner side. Thus, for example, also in the embodiment of FIGS. 1 to 5, the inner stiffening tube part 6 may be prefabricated on the outer side of the tube membrane 5. The flexible outer sleeve and/or the at least one inner member may also be formed in each case by a tubular casing with a cast-in coil spring or a cast-in tubular fabric or by a coil spring. This and similar designs of the flexible stiffening elements and the thereby achievable characteristics and advantages are explained in detail in the assignee's German patent applications DE 10 2006 007 974.4 and DE 10 2006 018 489.0 with respect to the tube described there, which can be stiffened in a controlled manner, and can be used in the same fashion for the present section V, whereby the content of these German patent applications are incorporated into the present application by reference in order to avoid unnecessary repetition.

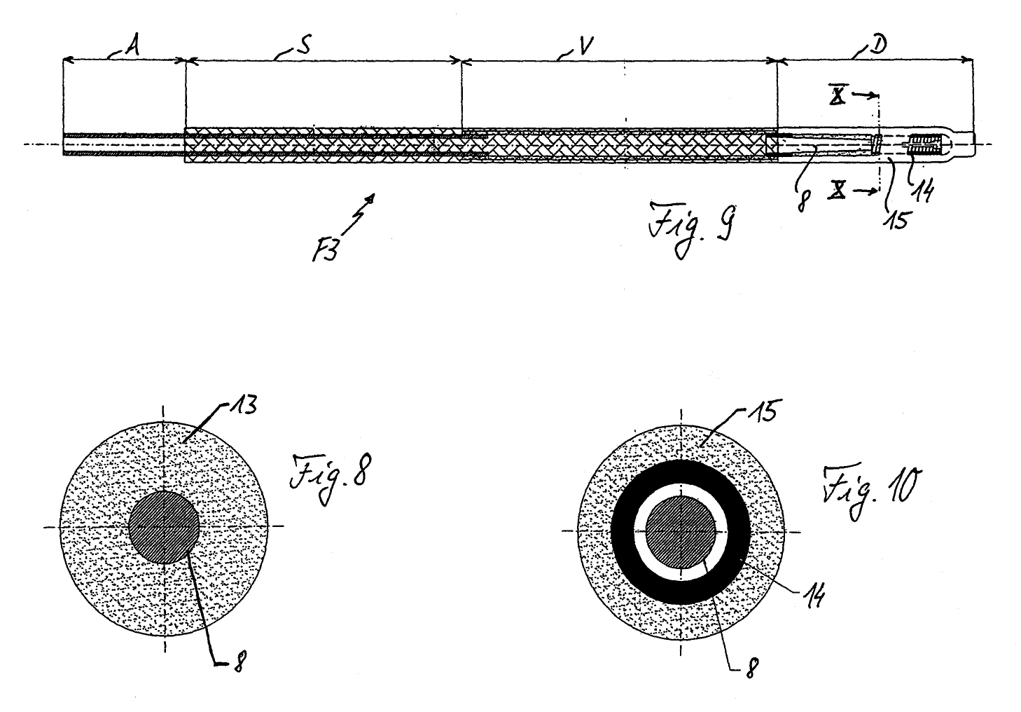

FIGS. 6 to 8 illustrate a second guide wire unit F2 as a variant of the first guide wire unit F1, which essentially corresponds to the latter, so that only the existing differences will be discussed in the following. Specifically, the second guide wire unit F2 is modified with respect to the first guide wire unit F1 in the stiff section S and in the distal end section D. In this example, in the stiff end section S, the pressure tube 1, instead of being surrounded by a coil spring, is surrounded by the structure consisting of the inner stiffening tube part 6 and the outer stiffening tube part 7. Since the pressure tube has a stiff design; i.e., under the effect of the stiffening pressure by the pressure medium, it does not yield to the radial pressure force FR, the inner and the outer stiffening tube part 6, 7 in the stiff section S only have an enveloping function and not the function of adjusting different stiffnesses existing in section V, which can be stiffened in a controlled fashion. With respect to manufacturing and design, there is the advantage that the structure consisting of the inner and the outer stiffening tube part 6, 7 may extend in one piece continuously by way of the stiff section S and Section V which can be stiffened in a controlled manner. In section V, which can be stiffened in a controlled manner, this complex carries out the function described above concerning the embodiment of FIGS. 1 to 5, for adjusting different stiffnesses, in that there, in the case of the acting stiffening pressure by the introduced pressure medium, the tube membrane 5 presses the inner stiffening tube part 6 radially toward the outside against the outer stiffening tube part 7.

In the case of the second guide wire unit F2, in the distal end section D, the distal guide wire core 8 is surrounded in a manner which is conventional per se by a solid plastic covering 13, for example, of a polyurethane material, as required, supplemented by an additional surface coating. In this embodiment, the plastic covering 13 simultaneously forms the atraumatic hemispherical distal end.

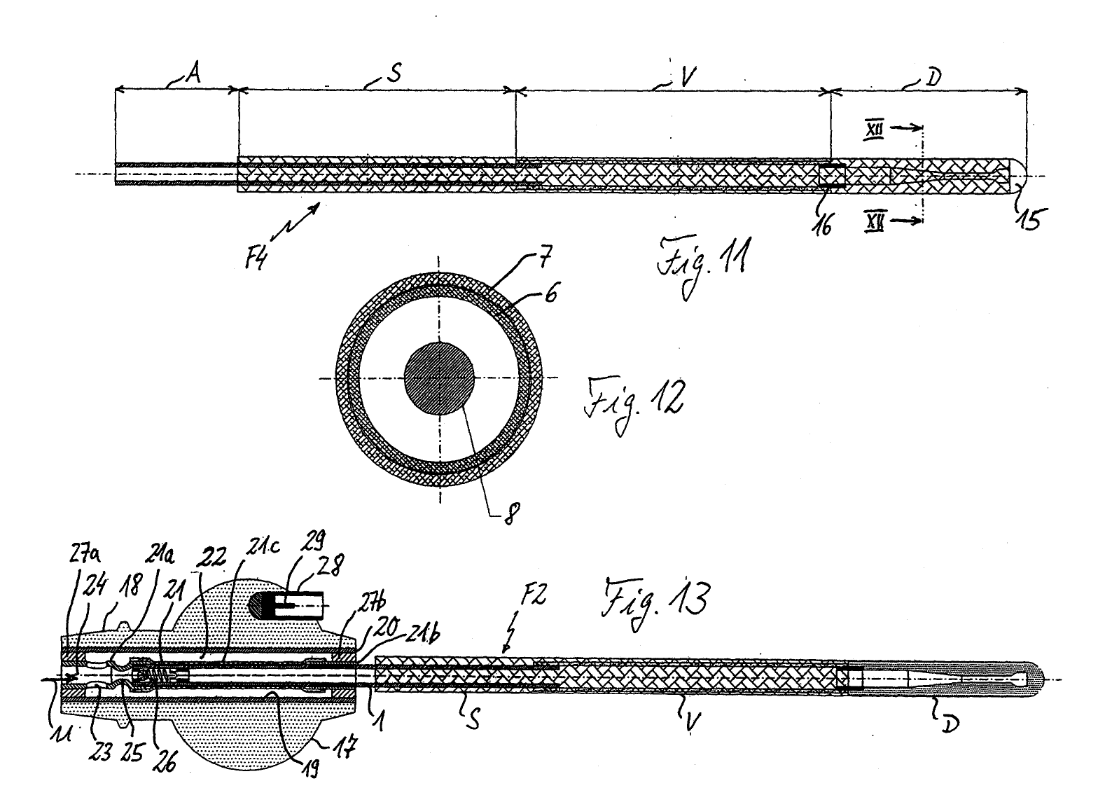

FIGS. 9 and 10 illustrate a third guide wire unit F3 as a variant of the second guide wire unit F2 of FIGS. 6 to 8, in which case, in the following, only the existing differences need to be discussed, which here are situated only in the distal end section D; that is, the three other sections A, S and V are identical with those of the second guide wire unit F2.

Especially in the case of the third guide wire unit F3, the distal guide wire core 8 is surrounded only in a forward tapered region by a coil spring 14 which, in turn, together with the rearward thicker part of the distal guide wire core 8, is surrounded by a plastic covering 15, for example, made of a polytetrafluor ethylene (PTFE) material. Again, as required, an additional surface coating may be provided. The coil spring 14 is preferably designed to be visible by x-ray, so that the positioning of the distal end section D of the guide wire unit in use in a patient's tissue duct can be followed by x-ray observation.

FIGS. 11 and 12 illustrate a fourth guide wire unit F4 as another variant of the second guide wire unit F2 of FIGS. 6 to 8. Again, only the existing differences need to be discussed in the following.

Specifically, the fourth guide wire unit F4 differs from the second guide wire unit F2 only with respect to the design of the distal end section D in that here the structure consisting of the inner stiffening tube part 6 and the outer stiffening tube part 7 extends also beyond the distal end section D. The fourth guide wire unit F4 therefore has a uniform enveloping or outer side design consisting of the tube braiding of the outer stiffening tube part 7 beyond the stiff section S, the section V which can be stiffened in a controlled manner and the distal end section D, which outer stiffening tube part 7 surrounds the tube braiding of the inner stiffening tube part 6. In addition to the design-related advantage, the manufacturing-related advantage of this variant should also be mentioned here. At the distal end, the guide wire unit F4 again ends with a hemispherical atraumatic end cap 15.

As in the stiff section S, in the distal end section D, the structure consisting of the inner and outer stiffening tube sections 6, 7 does not have the function of adjusting different stiffnesses, which exists only in section V that can be stiffened in a controlled manner. This is based on the above-mentioned fact that the transition area between section V, which can be stiffened in a controlled manner, and the distal end section D is closed off in a fluid-tight manner by way of the cylindrical connection piece 16, as explained above with respect to the first guide wire unit F1 and as, in addition, analogously is also the case with respect to the second and third guide wire unit F2, F3. The reason is that the construction of the cylindrical connection piece 16 with the distal guide wire core 8 distally mounted thereon and the tube membrane 5 held proximally fluid-tightly thereon, in each case, ensures the fluid-tight closure of section V, which can be stiffened in a controlled manner, with respect to the distal end section D. The difference between the individual illustrated embodiments therefore only consists of the fact that, in the case of the first guide wire unit F1, the distal enveloping coil spring 9 is fastened on the distal side of the connection piece 16, while, in the case of the second and third guide wire unit, the connection piece 16 is surrounded there by the plastic covering and, in the case of the fourth guide wire unit F4, by the structure consisting of the inner and outer stiffening tube part 6, 7.

FIG. 13 illustrates a proximal connection according to the invention for the example of the second guide wire unit F2, in which case also other guide wire units according to the invention can be connected in the same manner. As shown in FIG. 13, a connection body 17 derived from a conventional Luer connection is provided as the proximal connection, into which connection body 17 the guide wire unit F2 is fitted with its connection section A and is held in a releasable manner, on an opposite side, the connection body 17 having a conventionally shaped pressure connection piece 18.

The connection body 17 has a central hollow channel 19 into which, in a form-fitting manner, a cylindrical guide wire holding insert is placed, which has an outer sleeve 20 and an inner sleeve 21 forming an annular pressure chamber 22 between one another. The inner sleeve is formed in three parts by a proximal sleeve part 21a, a distal sleeve part 21b and a cylindrical sealing membrane 21c situated in-between and held by the sleeve parts. The proximal sleeve part 21a is provided with connection openings 23 between the annular pressure channel 22 and a central pressure medium feeding channel 24 and, in a distally adjoining area, forms a narrowing nozzle 25 for the central pressure medium channel 24. In its distal end region, the proximal sleeve part 21 receives the proximal end of the pressure tube 1 of the guide wire unit F2, a check valve 26 being inserted there into the pressure tube 1. The proximal sleeve part 21a and the distal sleeve part 21b are each supported on a sealing 27a, 27b, which bound the annular pressure chamber 22 in the axial direction. The cylindrical sealing membrane 21c is placed in a fluid-tight manner, for example, by shrink fitting, on the one hand, on the distal end region of the proximal sleeve part 21a and, on the other hand, on the proximal end region of the distal sleeve part 21b.

During the operation, when the pressure medium 11 is being fed, pressure in the annular pressure chamber 22 is at first built up more rapidly than in the inner channel downstream of the nozzle 25 in that the latter is selected with a narrower cross-section than the total cross-section of the connection openings 23. As a result, the sealing membrane 21c is rapidly and reliably pressed radially toward the inside against the inserted pressure tube 1, so that the pressure tube 1 is held in the connection body in a secure and fluid-tight manner. In the further course of the introduction of pressure medium, the desired pressure is built up in the guide wire unit F2 and particularly in its section V which can be stiffened in a controlled manner, in order to change the latter to a condition of increased stiffness. For this purpose, the pressure medium 11 flows through the nozzle 25 and the opening check valve 26 into the pressure tube 1 and from there farther into the pressure channel 4 of section V which can be stiffened in a controlled manner.

After the pressure buildup has taken place, the connection body 17 can be withdrawn from the not shown conventional pressure medium source; after which the check valve 26 closes so that the stiffening pressure in the guide wire unit F2 is maintained, while the pressure in the annular pressure channel is reduced so that the sealing membrane 21c is no longer pressed firmly against the pressure tube 1 and the connection body 17 can therefore be withdrawn from the pressure tube 1 as required.

In the illustrated embodiment, a pressure relief pin 28 is received in a receiving device at the connection body 17, which pressure relief pin 28 is designed such that it can be fitted onto the end of the pressure tube 1 equipped with the check valve 26, and, by means of a central mandrel 20 opens the check valve 26. In this manner, the pressure medium can be discharged again from the guide wire unit F2 and particularly from its pressure channel 4 in section V, which can be stiffened in a controlled manner, so that the guide wire unit F2 can be changed into a condition of lower stiffness. Concerning additional characteristics and advantages of this type of connection, reference can be made to the German patent application 10 2006 024 095.2, which was filed on May 17, 2006. The content of the German patent application is incorporated by reference herein in order to avoid unnecessary repetitions.

It is understood that the guide wire unit according to the invention can also be supplied with the necessary pressure medium, such as compressed air, by other conventional pressure connections. It is also understood that the invention can be used for guide wires for the positioning of catheters as well as guide wires in the case of other medical and non-medical uses wherever there is the requirement of being able to optionally adjust different stiffnesses for a guide wire unit at least along a portion of its length. In this respect, it is particularly understood that, in corresponding embodiments of the invention, the distal end region D and/or the stiff section S and/or the connection area A may be absent, and/or section V, which can be stiffened in a controlled manner, may extend along the entire length of the guide wire unit, and/or several sections which can be stiffened in a controllable manner in this fashion may be provided in different axial positions along the guide wire unit.

1.-17. (canceled)

18. A guide wire unit, comprising:
a first section stiffenable in a pressure-medium-controlled manner, the first section having a pressure channel for controllably generating a stiffening pressure by introducing a pressure medium;
wherein the first section has flexible stiffening elements which, under a pressure load when subjected to the stiffening pressure, are pressed against one another to increase a stiffening effect;
wherein the flexible stiffening elements comprise an inner stiffening tube part and an outer stiffening tube part surrounding the inner stiffening tube part, the outer stiffening tube part being radially less flexible than the inner stiffening tube part under the pressure load, whereby the inner stiffening tube part is pressed against an interior side of the outer stiffening tube part to increase the stiffening effect.

19. The guide wire unit according to claim 18, wherein at least one of the outer stiffening tube part and the inner stiffening tube part is formed by a braided tube.

20. The guide wire unit according to claim 18, further comprising at least one of:
a flexible distal end section disposed distally in front of the first section stiffenable in a controlled manner;
a stiff section which is disposed proximally in front of the first section stiffenable in a controlled manner, the stiff section including a pressure-resistant tube for guiding through the pressure medium.

21. The guide wire unit according to claim 19, further comprising at least one of:
a flexible distal end section disposed distally in front of the first section stiffenable in a controlled manner;
a stiff section which is disposed proximally in front of the first section stiffenable in a controlled manner, the stiff section including a pressure-resistant tube for guiding through the pressure medium.

22. The guide wire unit according to claim 18, further comprising:
a central guide wire core which extends in the first section, the pressure channel being constructed as an annular channel which surrounds the guide wire core radially on the outside.

23. The guide wire unit according to claim 20, further comprising:
a central guide wire core which extends in the first section, the pressure channel being constructed as an annular channel which surrounds the guide wire core radially on the outside.

24. The guide wire unit according to claim 18, wherein the flexible stiffening elements are operatively configured to frictionally engage and/or to form fit for the stiffening interaction.

25. The guide wire unit according to claim 18, wherein the pressure channel is radially bounded by a surrounding, radially flexible tube membrane, which is adjoined radially on the outside by the flexible stiffening elements.

26. The guide wire unit according to claim 25, further wherein the flexible stiffening elements comprise a flexible outer sleeve and at least one inner member arranged between the tube membrane and the outer sleeve, which inner member, under a pressure load, is pressed radially to the outside against the flexible outer sleeve by a radially outward-pointing pressure force component.

27. The guide wire unit according to claim 26, wherein several inner members extending with an axial direction component are arranged in a circumferential direction distributed between the tube membrane and the outer sleeve.

28. The guide wire unit according to claim 26, wherein at least one inner member is formed of one of: a flat or round wire material, a strand material, or a rope material, or of a braided tube.

29. The guide wire unit according to claim 26, wherein the flexible outer sleeve is formed by the outer stiffening tube part.

30. The guide wire unit according to claim 20, wherein the distal end section has a central distal guide wire core and a covering surrounding the core.

31. The guide wire unit according to claim 30, wherein the covering comprises at least one of a plastic coating, a coil spring, and a braided tube.

32. The guide wire unit according to claim 20, wherein the pressure-resistant tube is surrounded by a covering comprising a coil spring and/or a braided tube.

33. The guide wire unit according to claim 31, wherein the pressure-resistant tube is surrounded by a covering comprising a coil spring and/or a braided tube.

34. The guide wire unit according to claim 33, wherein the braided tube of the covering in the distal end section and/or the braided tube of the covering in the stiff section is formed in one piece with a braided tube of the outer stiffening tube part and/or with a braided tube of the inner stiffening tube part.

35. The guide wire unit according to claim 18, further comprising:
a proximal pressure medium connection device, which has a pressure medium connection tube in which a check valve is provided and/or which is releasably held by a tube holding device, which is activatable by admission of pressure, in a fluid-tight manner in a connection body.

36. The guide wire unit according to claim 35, wherein the tube holding device comprises a sealing membrane radially surrounding the pressure medium connection tube, which sealing membrane is adjoined radially on the outside in the connection body by an annular pressure channel.

37. The guide wire unit according to claim 36, wherein the annular pressure channel is acted upon by the same pressure medium as the first section which is stiffenable in a controlled manner.
